(19) United States
(12) Patent Application Publication
(10) Pub. No.: US 2011/0212111 A1
(43) Pub. Date: Sep. 1, 2011

(54) MHC II Associated Protein and Uses Thereof

(76) Inventors: John C. Cambier, Denver, CO (US); Lei Jin, Denver, CO (US)

(21) Appl. No.: 13/127,465

(22) PCT Filed: Nov. 2, 2009
(86) PCT No.: PCT/US09/62996

§ 371 Date: May 3, 2011

Related U.S. Application Data
(60) Provisional application No. 61/111,279, filed on Nov. 4, 2008.

(51) Int. Cl. A61K 39/395; C12N 9/02; C12Q 1/26; A61P 29/00; A61P 31/04; A61P 31/12

(52) U.S. Cl. 424/172.1; 435/189; 435/25

(57) ABSTRACT

The present invention provides a MHC II associated protein, MPYS, and methods for using the same. In particular, the MHC II-associated protein of the present invention mediates apoptosis and influences the susceptibility to infectious diseases. The present invention also provides methods for treating clinical conditions, methods for identifying therapeutically useful compounds using such a protein, and methods for determining susceptibility to infectious disease.

### CROSS-REFERENCE TO RELATED APPLICATIONS

This application claims the priority benefit of U.S. Provisional Application No. 61/111,279, filed Nov. 4, 2008, which is incorporated herein by reference in its entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of Grant No. RO1 AI020619 awarded by the National Institutes of Health.

### FIELD OF THE INVENTION

The present invention relates to a MHC II associated protein, methods for using the same for treating various clinical conditions and for identifying therapeutically useful compounds, as well as methods predicting and diagnosing susceptibility to an infectious disease.

### BACKGROUND OF THE INVENTION

Anti-MHC II mAbs have advanced to Phase I (1D09C3) or phase II (Hu1D10) clinical trials for refractory and relapsed non-Hodgkin's lymphoma (NHL), and relapsed low-grade or follicular lymphoma. Cell death induced by anti-MHC II mAbs is independent of complement, Fc receptors and caspase activation, and occurs by unknown mechanisms.

Although one of the well known functions of type II Major Histocompatibility Complex (MHC II) is presentation of antigenic peptides to T lymphocytes, these molecules can also transduce signals leading alternatively to cell activation or apoptotic death. MHC II is a heterodimer of two transmembrane proteins each containing a short cytoplasmic tail that is dispensable for transduction of death signals. This suggests the function of an undefined MHC II associated transducer in signaling the cell death response.

The ability of MHC II to transmit signals was first recognized more than twenty years ago. Depending on species, cell type and cell activation state, anti-MHC II mAb stimulation induces tyrosine phosphorylation, calcium mobilization, cAMP production, MAPK, AKT and PKC activation. It has been shown that in activated murine B cells, MHC class II signals trigger tyrosine phosphorylation and calcium mobilization via associated CD79a and b. These responses are also shown to be induced by TCR binding to the MHC II/antigenic peptide complexes on B cells.

Several recent studies have shown biologic importance of MHC II signaling; particularly MHC II-mediated death signaling. For example, it has been shown that cros slinking of MHC II by mAb, as well as via cognate MHC II-TCR interaction, can lead to Fas independent APC death. It has also been shown that dendritic cells (DCs) undergo accelerated clearance from the lymphoid organs after interacting with antigen-specific T cells. Moreover, it has been shown that prolonged DC survival can lead to autoimmunity. Thus, without being bound by any theory, it is believed that MHC II-mediated death signaling functions to limit potentially dangerous uncontrolled immune responses by elimination of APCs after they have served their antigen presenting purpose. Given the similar response of certain B cells to MHC II aggregation, it is believed that under certain circumstances they too are eliminated by this mechanism.

The molecular basis of MHC II-mediated signaling of cell death is unknown. However this response has been shown to be independent of complement and Fc receptors. Effective humanized anti-MHC II mAbs are believed to be IgG4 isotype antibodies that are substantially devoid of CDC (complement-dependent cytotoxicity) and do not mediate ADCC (antibody dependent cell mediated cytotoxicity). All of these mAbs have been shown to induce death in lymphoma/leukemia tumor cells. Thus, death induced by these antibodies, and by extension T cell antigen receptors, is believed to be mediated by MHC II signaling. Furthermore, it has been shown that protein kinase C activation is required for MHC II-mediated death in Raji human B cell lymphoma, mature dendritic cells (DC) and activated THP-1 monocytes, but not in primary human plasmacytoid DCs. MHC II-mediated signaling of death also appears to occur independent of Src-family kinase and caspase activation in Raji and Ramos cells. Reactive oxygen species (ROS) production and JNK activation have been implicated in MHC II mediated death in lymphoma line JVM-2 and GRANTA-519. Thus, data are fragmentary and there is no consensus regarding how MHC class II molecules transduce death signals. Knowing the mechanism of how MHC class II molecules transduce death signals will lead to a new therapeutic target in various clinical conditions as well as aid in identifying therapeutically useful compounds.

Accordingly, there is a need for identifying how MHC class II molecules transduce death signals and using such knowledge for various therapeutic applications, including finding therapeutic compounds useful in treating conditions in which MPYS is expressed in cells mediating said condition.

### SUMMARY OF THE INVENTION

Some aspects of the invention provide an isolated peptide having at least 80% homology with the sequence of SEQ ID NO:1, or a gene or a nucleic acid sequence encoding such a protein. The protein encoded by sequence of SEQ ID NO:1 and variants thereof is termed MPYS (or MPHS in human) protein. In some embodiments, the isolated peptide has at least 90% homology, often the isolated peptide has at least 95% homology and more often at least 99% homology with the sequence of SEQ ID NO:1.

Other aspects of the invention provide methods for identifying a compound with a potential for treating a clinical condition mediated by cells expressing MPYS. Such methods comprise:
contacting a test compound with a cell that expresses gene or expresses a transcript of gene that encodes a protein associated with a peptide sequence of SEQ ID NO:1; and
identifying the test compound that modulates the expression of the gene or the transcript of the gene that encodes the protein associated with the peptide sequence of SEQ ID NO:1.
   Modulation of the expression of the gene or the transcript of the gene that encodes the protein associated with the peptide sequence of SEQ ID NO:1 is indicative that the compound has a potential for treating the clinical condition mediated by cells expressing MPYS.

In some embodiments, the clinical condition comprises tumor, inflammation, autoimmunity, immunodeficiency immunosuppression, or a combination thereof. Exemplary MPHS expressing tumors include, but are not limited to, leukemia, lymphoma and mastocytoma.

In other embodiments, an increase in the expression of the gene or the transcript of the gene is indicative that the compound has a potential for treating the clinical condition.

Still in other embodiments, decrease in the expression of the gene or the transcript of the gene is indicative that the compound has a potential for treating the clinical condition.

Still other aspects of the invention provide methods for identifying a compound with a potential for treating a clinical condition in which a cell expressing MPHS is required for maintenance of the clinical condition. Such methods typically comprise:
contacting a test compound with a protein or a cell comprising a protein having at least 80% homology with MPHS protein; and
identifying a compounds that modulates the protein activity.
   Modulation of the protein activity is indicative that the compound has a potential to treat the clinical in which a cell expressing MPHS is required for maintenance of the clinical condition. Exemplary protein activities include, but are not limited to, induction of cell death, suppression of immunity, etc.

In some embodiments, increase in the protein activity is indicative that the compound has a potential for treating the clinical condition. Exemplary clinical conditions that can be treated by increasing the MPHS protein activity include, but are not limited to, tumor (such as leukemia, lymphoma and mastocytoma), inflammation, and autoimmunity.

Yet other aspects of the invention provide methods for treating a clinical condition in which increased MPHS activity (relative to normal or control) causes the illness. Such methods comprise administering to the subject a therapeutically effective amount of a compound that reduces the expression of MPHS protein or the activity of MPHS protein. Exemplary clinical conditions that can be treated by reducing the expression of MPHS protein or the activity of MPHS protein include, but are not limited to, those described herein as well as immunodeficiency and immunosuppression clinical conditions.

In some embodiments, the compound enhances immunization. In other embodiments, the compound can also function as an adjuvant.

Yet in some embodiments, methods of the invention comprise inhibiting or reducing the activity of MPHS protein. In other embodiments, methods of the invention comprise reducing the expression of MPHS protein. Reduction of the expression of MPHS protein can be achieved by any one of the methods known to one skilled in the art including using a siRNA.

Still in other embodiments, the compound inhibits transcription of the MPHS gene. While in some embodiments the compound inhibits translation of MPHS mRNA.

Yet other aspects of the invention provide methods for treating a clinical condition in which reduced MPHS activity (relative to normal or control) causes the illness. Such methods comprise administering to the subject a therapeutically effective amount of a compound that increases the expression of MPHS protein or the activity of MPHS protein. Exemplary clinical conditions that can be treated by increasing the expression of MPHS protein or the activity of MPHS protein include, but are not limited to, tumor (such as leukemia, lymphoma and mastocytoma), inflammation, and autoimmunity.

In some embodiments, the compound enhances apoptosis. In other embodiments, the compound can also function as an adjuvant. Still in other embodiments, the compound facilitates the adjuvant activity of a DNA vaccine.

Yet in some embodiments, methods of the invention comprise increasing the activity of MPHS protein. In other embodiments, methods of the invention comprise increasing the expression of MPHS protein.

Still in other embodiments, the compound promotes or facilitates transcription of the MPHS gene.

Yet some aspects of the invention provide a therapeutic use for an isolated antibody specific for a protein that is at least 80% homologous to a peptide sequence of SEQ ID NO:1.

Still other aspects of the invention provide methods for determining susceptibility of a subject to an infectious disease. Typically, such methods comprise determining the nucleotide base or the corresponding amino acid residue of a single nucleotide polymorphism consisting of rs11554776, rs1131769, rs7380824, or a combination thereof. The presence of the amino acid residue or the nucleotide base that corresponds to coding of the amino acid residue histidine at rs11554776 or rs1131769, or the amino acid residue glutamine at rs7380824, or a combination thereof is indicative that the subject is more susceptible to an infectious disease relative to a subject that has the amino acid residue or the nucleotide base that corresponds to coding of the amino acid residue arginine at rs11554774, rs1131769, and rs7380824.

In some embodiments, such methods comprise determining the nucleotide base, for example, using any one of the PCR based technologies.

In other embodiments, such methods comprise determining the amino acid residue that corresponds to SNP rs11554776, rs1131769, rs7380824, or a combination thereof.

Yet in other embodiments, the infectious disease comprises a disease mediated by a virus (such as influenza virus, hepatitis virus, human immunodeficiency virus, adenoviruses, vaccinia virus, cytomegalovirus, or HSV), or bacteria (such as Listeria monocytogenies, or any gram negative pathogen), or a combination thereof. Still in other embodiments, methods and compositions of the invention reduce or prevent susceptibility to gram positive bacteria (e.g., L. monocytogense and Mycobacterium tuberculosis) and gram negative bacteria (e.g., sepsis response in human).

### BRIEF DESCRIPTION OF THE DRAWINGS

FIG. 1 shows alignment of mouse and human MPYS orthologs with annotation of their signaling motifs;

FIG. 2 is a schematic illustration of plasma membrane disposition of MPYS and its four transmembrane domains. Annotated are some of the residues that are conserved between human MPYS (i.e., MPHS) and mouse MPYS;

FIG. 3 is a schematic representation of dimerization of MPYS upon oxidation.

### DETAILED DESCRIPTION OF THE INVENTION

Some aspects of the invention provide identification and cloning of a MHC II associated protein (referred herein as MPYS protein or MPHS protein when referring only to the corresponding human protein) that contains four putative transmembrane (TM) domains, an extended cytoplasmic tail and multiple protein interaction motifs. Found in plasma membrane and mitochondria of lymphocytes and myeloid cells, MPYS can be tyrosine phosphorylated and associates with the SH2-containing inositol 5-phosphatase SHIP1 and the phosphotyrosine phosphatase SHP1. Reduction of MPYS expression by >90% using short-hairpin RNA (shRNA) significantly reduced anti-MHC II mAb-induced death of lymphoma cells. Thus, it is believed that MPYS functions in promotion of cell death.

The plasma membrane tetraspanner of the present invention (e.g., MPYS) is associated with MHC II and mediates its transduction of death signals. MPYS is unusual among tetraspanners in containing an extended c-terminal cytoplasmic tail (˜200aa) with multiple embedded signaling motifs. MPYS is tyrosine phosphorylated upon MHC II aggregation and associates with inositol lipid and tyrosine phosphatases. It is believed that in some instances MHC class II-mediated cell death signaling requires MPYS-dependent activation of the ERK signaling pathway.

The ability to mediate cell death signaling makes MHC II a candidate therapeutic target for treatment of certain malignancies. Currently, anti-MHC II mAbs (1D09C3, Hu1D10) are being tested in clinical trials involving patients with refractory and relapsed non-Hodgkin's lymphoma or relapsed low-grade or follicular lymphoma. These anti-MHC II mAbs exhibit rapid and potent in vitro tumoricidal activity for lymphoma/leukemia cells with no significantly observable long-lasting hematological toxicity in primates.

In accordance with the present invention, an isolated peptide, or an isolated protein, is a peptide or protein that has been removed from its natural milieu (i.e., that has been subject to human manipulation), its natural milieu being the cell or membrane in which the peptide or protein is found in nature. As such, “isolated” does not necessarily reflect the extent to which the peptide or protein has been purified, but indicates that the molecule does not include other components of the cell in which the peptide or protein is found in nature. In some cases, an isolated peptide or protein of the present invention is produced using molecular cloning (e.g., by recombinant DNA technology) or chemical synthesis.

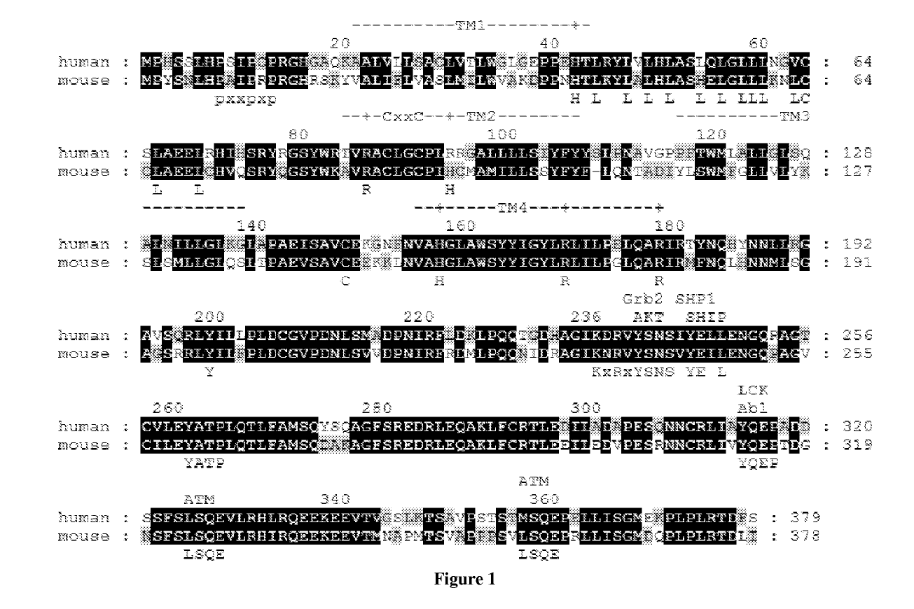

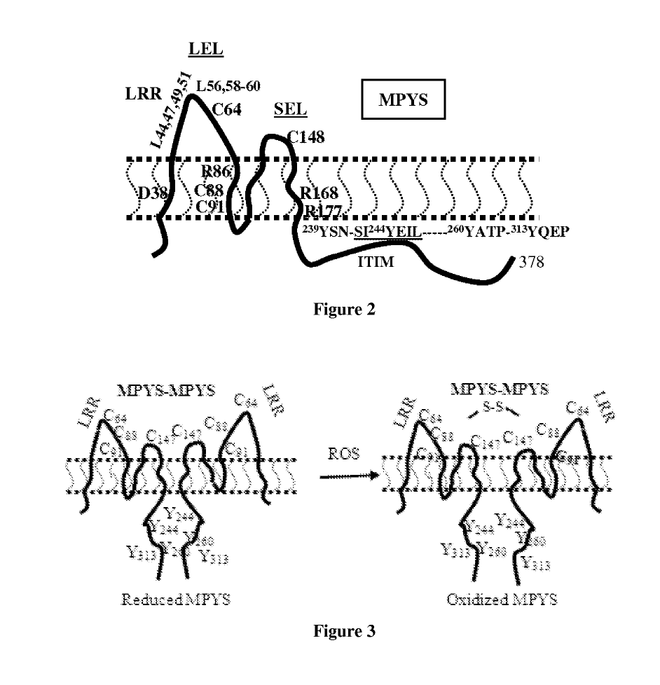

The minimum size of a peptide or protein of the present invention is a size sufficient such that the peptide or protein has a desired biological activity, for example, sufficient to be used as a target in an assay or in any therapeutic method discussed herein. The minimum size of a peptide (i.e., number of amino acids) that is useful has at least about 50% of the contiguous number of amino acids listed in the sequence shown in FIG. 1. Typically, the minimum size of a peptide that is useful has at least about 70%, often at least about 80%, more often at least 90%, and still more often at least about 95% of the contiguous number of amino acids listed in the sequence shown in FIG. 1. It should be appreciated that MPYS protein sequence shown in FIG. 1 does not represent all of the isoforms or mutants. Accordingly, the scope of the present invention includes all isoforms and mutants of MPYS protein sequence shown in FIG. 1.

Other aspects of the invention provide antibodies to the peptide or protein shown in FIG. 1. Such antibodies are useful in a wide variety of therapeutic applications including treating clinical conditions in which a cell expressing MPYS is required for maintenance of the clinical condition. Such antibodies are expected to induce the death of the offendingcells. Such antibodies might also be useful in treating diseases caused by overexpression of MPYS or higher than normal MPYS activity. In this case antibody treatment may reduce activity by reducing protein level or activity.

Some aspects of the invention relate to methods for identifying compounds that modulate the expression or activity of the protein comprising a peptide sequence shown in FIG. 1. Such compounds can be used to further study mechanisms associated with various MHC II complex functions or more typically, serve as a therapeutic agent for use in the treatment or prevention of at least one symptom or aspect of clinical conditions in which a cell expressing MPYS is required for maintenance of the clinical condition. An assay can be used for screening and selecting a chemical compound or a biological compound having regulatory activity as a candidate reagent or therapeutic based on the ability of the compound to regulate the expression or activity of the protein comprising the peptide sequence shown in FIG. 1. Reference herein to regulating the protein comprising the peptide sequence shown in FIG. 1 can refer to regulating transcription of the gene encoding the protein comprising the peptide sequence shown in FIG. 1 and/or regulating the translation and/or activity of the protein comprising the peptide sequence shown in FIG. 1. A regulation of the expression of the protein comprising the peptide sequence shown in FIG. 1 or regulation of the activities of the protein comprising the peptide sequence shown in FIG. 1 (e.g., biological activity) can be used to identify a therapeutic compound. Therapeutic compounds identified in this manner can then be re-tested, if desired, in other assays to confirm their activities with regard to the protein comprising the peptide sequence shown in FIG. 1 or a cellular or other activity related thereto.

### Antibodies

Means for preparing and characterizing antibodies are well known in the art (See, e.g., Antibodies: A Laboratory Manual, Cold Spring Harbor Laboratory, 1988; incorporated herein by reference in its entirety). Methods for generating polyclonal antibodies are well known in the art. Briefly, a polyclonal antibody is prepared by immunizing an animal with an immunogenic composition and collecting antisera from that immunized animal. A wide range of animal species may be used for the production of antisera. Typically the animal used for production of anti-antisera is a rabbit, a mouse, a rat, a hamster, a guinea pig or a goat. Because of the relatively large blood volume of rabbits, a rabbit is typically used for production of polyclonal antibodies.

A given composition may vary in its immunogenicity. Boosting the host immune system may be needed, as may be achieved by coupling a peptide or polypeptide immunogen to a carrier. Exemplary and typical carriers are keyhole limpet hemocyanin (KLH) and bovine serum albumin (BSA). Other albumins such as ovalbumin, mouse serum albumin or rabbit serum albumin may also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition may be enhanced by the use of non-specific stimulators of the immune response, known as adjuvants. Exemplary adjuvants can include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes may be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal may be bled and the serum isolated and stored, and/or the animal may be used to generate MAbs. For production of rabbit polyclonal antibodies, the animal may be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix.

It is contemplated herein that any antibody generated against any biomarker for disease may be humanized or designed for the particular subject being tested for disease or disease progression. For example, any known method for humanization of antibodies is contemplated.

Monoclonal antibodies (MAbs) may be readily prepared through use of techniques known in the art. Methods for generating monoclonal antibodies (MAbs) may begin the same as those for preparing polyclonal antibodies. Rodents such as mice and rats are typically used animals, however, the use of rabbit, sheep or frog cells is also possible. The use of rats may provide certain advantages, but mice are often used, with the BALB/c mouse being most often utilized as this is most routinely used and generally gives a higher percentage of stable fusions.

The animals are injected with antigen as described above. The antigen may be coupled to carrier molecules, such as keyhole limpet hemocyanin. The antigen may be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), can be selected for use in the MAb generating protocol. These cells may be obtained from biopsied spleens, tonsils or lymph nodes, or from a peripheral blood sample. Often, a panel of animals can be immunized and the spleen of the animal with the highest antibody titer will be removed and the spleen lymphocytes obtained by homogenizing the spleen with a syringe.

Antibody-producing B lymphocytes from the immunized animal can be fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. Any method known in the art for cell fusion is contemplated. Only cells that survive in selective media are used. Selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like. Selected hybridomas can be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines may also be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies may also be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

Fragments of the monoclonal antibody may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer. The monoclonal conjugates are prepared by methods known in the art. Other moieties to which antibodies may be conjugated include radionuclides such as ³H, ¹²⁵I, ¹³¹I ³²P, ³⁵S, ¹⁴C, ⁵¹Cr, ³⁶Cl, ⁵⁷Co, ⁵⁸Co, ⁵⁹Fe, ⁷⁵Se, ¹⁵²Eu, and ^{99m}Tc. Radioactively labeled monoclonal antibodies of the present invention are produced according to well-known methods in the art. For instance, monoclonal antibodies may be iodinated by contact with sodium or potassium iodide and a chemical oxidizing agent such as sodium hypochlorite, or an enzymatic oxidizing agent, such as lactoperoxidase. Monoclonal antibodies may be labeled with technetium-⁹⁹ by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody.

It will be appreciated by those of skill in the art that monoclonal or polyclonal antibodies specific for the protein comprising peptide sequence shown in FIG. 1 will have utility in several types of applications. These may include treating clinical conditions in which a cell expressing MPHS is required for maintenance of the clinical condition.

### MPYS and Susceptibility to Infectious Disease

MPYS plays an important role in immune response to infectious disease, e.g., anti-vial and anti-bacterial responses. The present inventors have discovered that unlike VISA (also called MAVS, IPS-1 and Card-IF), MPYS by itself only weakly activates IFNβ promoter. Without being bound by any theory, it is believed that this is due to MPYS's inability to activate AP-1 and only weak activation of NFκB. However, MPYS does potently activate ISRE (IFNβ-sensitive response elements) through Tyk2/STAT1 and TBK1/IRF3 pathways. MPYS can also enhance and B-DNA induced IFNβ response. MPYS induced IFNβ activation can be inhibited by LPS treatment and a small percentage of MPYS is associated with TLR4. The present inventors have also found that over-expressed MPYS is strongly associated and phosphorylated by TBK1. Furthermore, the present inventors had discovered that the three validated single nucleotide polymorphisms (SNPs) in human MPHS have altered ISRE and IFNβ activities showing the role of MPHS in host defense.

It is known that in addition to the acquired immunity, immune system recognizes pathogens through a limited number of germLine encoded pattern-recognition receptors (PRRs). These PRRs recognize microbial components, known as pathogen-associated molecular patterns (PAMPs) on cell surface (e.g., toll-like receptors: TLR1, 2, 4, 5, and 6), in endosome/lysome (TLR3, 7, 8, 9) or in the cytoplasm (NOD1,2, RIG-I/MDA5). These PRRs have different specificities with TLR4 complex for LPS, TLR5 for flagellin, TLR7,8 for ssRNA, TLR9 for bacterial unmethylated CpG, TLR3 for dsRNA and RIG-I/MDA5 for cytosolic viral RNA. However, the cytosolic sensor for mammalian dsDNA, also known as B-DNA, is still unknown.

The source for cytosolic dsDNA can come from damaged host cells, or viral infection such as adenoviruses, vaccinia virus, cytomegalovirus, HSV, Listeria sp., and Legionella sp. Thus, the cytoplasmic DNA sensors in some cases are connected to not only the host defenses but also to the pathogenesis of autoimmune diseases. It is known that TBK1 and IRF3 but not RIG-I, VISA or TLR9 is required for cytosolic dsDNA induced immune response.

As discussed above, the present inventors have discovered that MPYS is important in MHC II mediated apoptotic signaling. As exemplified in the Examples section, the present inventors used human 293GT cells and murine RAW264.7 macrophage cells to further show that MPYS activates the ISRE through Tyk2/STAT1 and TBK1/IRF3 pathway and expression of MPYS enhances B-DNA induced IFNβ production.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Abbreviations

MHC II=major histocompatibility complex II; ERK=extracellular signal regulated kinase; SHP-1=Src homology domain 2 (SH2)-containing tyrosine phosphatase-1; SHIP=SH2-containing inositol 5-pho sphatase; DC=dendritic cells; ITIM=immune tyrosine inhibitory motif; PI=propidium iodide; shRNA=short-hairpin RNA; Nano-LC/MS/MS=nanoscale liquid chromatography tandem mass spectrometry; APCs=antigen presenting cells.

### Example 1

### MPYS and Apoptosis

Induction and assay of Cell death

K46 cells were suspended in 5% complete IMDM media at a concentration of 10⁶ cells/mL and then transferred to round bottomed 96-well plates (100 μL/well) and cultured at 37° C. for 1 h. Biotinylated MHC II mAbs were added into the wells and incubated for 12 min. Avidin was then added into the wells and cells were cultured for the indicated time. PI (2 μg/mL) and Annexin V-Alexa Fluor 488 (1 μL/100 μL cells) (A-13201, Molecular Probes), or DiOC6 (50 nM) and PI (2 μg/mL) was used to measure apoptotic or dead cells by flow cytometry.

ERK inhibitor PD98059 (Calbiochem, 513000), AKT inhibitor LY294002 (Calbiochem, 440202), and p38 inhibitor SB203580 (Calbiochem, 559389) were used in this study. The inhibitor was added to cells at 5×10⁶ cells/mL and cultured for 4 hrs. Samples were then split into 1×10⁶ cells/mL and cell death was induced and measured as above. To inhibit src kinase, PP2 (Calbiochem, 529573) was added into cells and cultured for 30 min before cell death was induced as above.

### Immunoprecipitation and Immunoblotting

Cells were lysed in 0.33% CHAPS buffer (150 mM NaCl, 10 mM Tris pH 7.5, 10 mM sodium pyrophosphate, 2 mM Na₃VO₄, 10 mM NaF, 0.4 mM EDTA, 1 mM PMSF, and 1 μg/mL each of aprotinin, α₁-antitrypsin, and leupeptin) (40×10⁶/mL) at 4° C. for 1.5 h or overnight. Cell lysates were centrifuged at 12,000 g at 4° C. for 20 min. Supernatants were analyzed on a 10% SDS-PAGE gel. The gel was transferred to a PVDF membrane and proteins were probed with Abs and visualized using an ODYSSEY Infrared Imaging System (LI-COR). The Abs used were 4G10 phospho-tyrosine Ab, anti-HA mAb 16B12 (Covance Research, MMS-101P), anti-actin Ab (1-19) (Santa Cruz, sc1616), anti-SHP-1 Ab (12660), anti-SHIP Ab, anti-CD22 Ab (12045), anti-JNK 1 Ab (c-17) (Santa Cruz, sc474), anti-pan ERK (Transduction lab, 610123), and Abs from Cell Signaling Technology: anti-p38 (#9212), anti-p-P38 (#9211), anti-pERK (#9101), anti-AKT (#9272), anti-pJNK (#9251) and anti-pSer473-AKT (#9271).

Analysis of Intracellular Free Calcium Concentration ([Ca⁺²]ᵢ)

Cells were loaded with Indo-1AM (Molecular Probes, Eugene, Oreg.) for 30 min at 37° C. as recommended by the manufacturer. Anti-MHC II mAb (10 μg/mL) was then added to the suspension for another 12 min at 37° C. before cells were washed and suspended in IMDM supplemented with 2% FCS. The cells (10⁶ cells/mL) were analyzed before and after stimulation via cros slinking with avidin (20 μg/mL). Data were analyzed by Flow-Jo software (Tree Star, Inc., San Carlos, Calif.).

### Nano-LC/MS/MS

K46 cells (˜5×10⁸) were lysed in 0.33% CHAPS buffer and immunoprecipitation was performed as described by Lang et al. in Science, 2001, 291, 1537-40. Proteins were eluted from Ab-bound beads with 0.1 M citric acid (pH 2.0). Eluates in citric acid were neutralized with 2 M Tris (pH 10.0). The combined eluates were buffer exchanged into 25 mM ammonium bicarbonate (Fluka, 40867) using Zeba™ Desalt Spin Columns (Pierce, 89890). The protein ammonium bicarbonate solution was dried, reconstituted in 8.0 M urea (Sigma, U-4883), reduced in DTT (50 mM, Sigma D5545), alkylated with iodoacetamide (100 mM, Sigma, 11149) and trypsin (1 μg, Promega, V511A) digested overnight at 37° C. The digests were analyzed by reverse phase nanospray LC-MS/MS (Agilent 1100 HPLC, Agilent Ultra Ion Trap). Proteins were identified using the Spectrum Mill (Agilent) database search algorithm.

### Molecular Cloning of the Murine MPYS Gene

Primers (MPY5 and MPY3) were designed to amplify the full-length MPYS sequence from a K46 cDNA library (Table S2). PCR was carried out using Ex Tag™ DNA polymerase (TaKaRa, RR001) and sequenced.

Making MPYS-EGFP, MPYS-HA and MPYS-Flag constructs

Primers (MPYS-EGFP-For and Rev) were designed to include restriction enzyme sites into the end of the MPYS coding sequence. The PCR product was cloned into the EcoR I-Not I sites of a pMXI-EGFP fusion construct. A reverse primer (HA-MPY-Rev) was designed to include a Xho I site and an HA tag sequence. HA-MPYS was amplified using the primers MPYS-EGFP-For and HA-MPY-Rev. The PCR product was cloned into EcoR I-Xho I sites of a pMXI-IRES-EGFP vector. For, LEL-Flag construct, a flag sequence was inserted between V⁷³ and Q⁷⁴ of MPYS. For SEL-Flag construct, the flag sequence was inserted between E¹⁴⁹ and K¹⁵⁰ of MPYS. The construct was confirmed by sequencing.

### Generation of Polyclonal Anti-MPYS abs

Primers (GST-MPYS-For and Rev) were designed to amplify cDNA sequence encoding the C-terminal 101 aa of MPYS. The PCR product was cloned into the BamH I-Not I sites of pGEX-5X-1 (Amersham Biosciences, 27-4585-01) to make the peptide, which was used to immunize a rabbit. The polyclonal Ab was affinity purified using peptide-coupled Sepharose™ beads.

shRNA Knock-Down of MPYS Expression

Two shRNAs were designed to target sequences in exon 5 and 7 of MPYS using pSicoOligomaker 1.5. The forward and reverse primers (1 μL each of 1 μg/μL) were annealed and the annealed oligos were ligated into pLL3.7 through Hpa I and Xho I sites. A shRNA that targets luciferase gene was used as control. Lentiviruses were generated as described by Ventura et al. in Proc Natl Acad Sci USA, 2004, 101, 10380-5.

### Cell Surface Biotinylation

Cells (25×10⁶ cells/mL) were suspended in PBS. Sulfo-NHS-LC-Biotin (Pierce, 21335) (0.5 mg/mL) was added and cells were incubated with rotation for 30 min at RT. Glycine (50 mM) was added into the cell suspension for an additional 5 min to quench excess succinimidyl ester. The cells were then centrifuged and lysed in 0.33% CHAPS buffer.

### Live-Cell Imaging

K46 cells expressing MPYS-GFP (5×10⁴) were loaded into Lab-Tek chamber (177445, Nunc, Denmark) slides and allowed to adhere for 12 hr. Cell were loaded with 2 μg/mL Hoechst 34580 (H21486, Invitrogen) and 1 μM dihydrorhodamine 6G (D633, Invitrogen) for 30 minutes. Images were collected using a 63× objective in an inverted Zeiss 200M microscope.

Proteins in the MHC II complex with at least two unique peptides were identified by nano-LC/MS/MS. In particular, peptides from MHC II α/β chains, CD20, CD37 and MPYS were identified from the mass spectra of the MHC II complex and MHC II α/β chains, and MPYS in MPYS complex.

### Results

### MHC II mAb Induces Time and Dosage Dependent Death of K46 B Lymphoma Cells

MHC II induced cell death in a murine B lymphoma line K46 was studied. Crosslinking MHC II by mAbs in K46 cells has been shown to induce tyrosine phosphorylation, calcium flux and PI-3K activation. MHC II crosslinking by biotinylated I-A^{b,d,q}/E^{d,k} reactive mAb M5/114 (rat IgG_{2b}) and avidin induced time and dosage dependent K46 B lymphoma exposure of phosphatidylserine as indicated by annexin V staining. The anti-Fc receptor mAb 2.4G2 (rat IgG_{2b}) was used as an isotype control. This MHC II mAb response, which indicates commitment to cell death, was observed as early as 1 hr and peaked at 5 hr. The cell death response was also measured by dual staining with propidium iodode (PI) and DiOC6(3)/PI. PI staining reflects cell permeability, and loss of DiOC6(3) staining reflects mitochondrial membrane depolarization. The control 2.4G2 mAb did not induce cell death. These data show that MHC II molecules transduce apoptotic cell death signals in K46 cells and this response is believed to involve depolarization of mitochondrial membranes.

PP2 Inhibits MHC II mAb-Induced Tyrosine Phosphorylation and Calcium Mobilization but not the Death Response.

Cross-linking MHC II by mAbs in K46 cells has been shown to induce protein tyrosine phosphorylation and calcium flux. To explore whether these signaling events were involved in the death response, the effect of the src kinase inhibitor PP2 on induction of cell death was assessed. MHC II mAb-induced tyrosine phosphorylation and calcium responses were blocked by PP2 treatment. However, MHC II mAb induced cell death remained largely intact after treatment with inhibitor. This indicates that tyrosine phosphorylation and calcium flux are not required for MHC II-mediated death response of human B cells.

MHC II mAb-Induced ERK Activation, but not AKT and p38 Activation, is Required for the Death Response

MHC II aggregation can lead to activation of AKT and MAPK kinases ERK, p38 and JNK in monocytes and human B cells. Whether these signaling events are activated upon MHC II mAb stimulation of K46 cells was investigated. Crosslinking MHC II by biotinylated M5/114 and avidin induced strong and sustained ERK and p38 activation in K46 cells. AKT was weakly activated by MHC II crosslinking as measured using phospho-AKT Ab blotting. There was weak basal JNK activation measured by phosphor-JNK Ab. However, MHC II activation did not increase JNK phosphorylation. Thus, it appears MHC II crosslinking activated AKT, ERK and p38 but not JNK in K46 cells.

Requirements for these signaling events in the cell death induced by MHC II mAb were also studied. Treatment of K46 cells with LY294002 inhibited MHC II mediated AKT activation but had no significant effect on the death response. MHC II-mediated p38 activation appeared to be completely inhibited by SB203580, but the inhibitor also had no significant effect on the death response. At 50 μM, ERK specific inhibitor PD98059 inhibited MHC II mediated cell death by about 50%. However, this dose did not completely inhibit MHC II induced ERK activation, which explains the residual cell survival and implicates ERK in the cell death response.

Although crosslinking MHC II with mAb leads to activation of AKT, ERK and p38, it appears only ERK activation is responsible for the death response of these cells.

### Identification of a Novel MHC II-Associated Membrane Protein Using Nanoflow Liquid Chromatography Tandem Mass Spectrometry (Nano-LC/MS/MS)

The signaling circuitry by which MHC II aggregation activates ERK is currently unknown. MHC II α and β chains contain cytoplasmic tails of only 12 and 18 amino acids, respectively, and deletion of these tails did not significatnly affect the death response. Thus, without being bound by any theory, it is believed that MHC II transmits death signals through an associated cell surface protein(s). B cell specific proteins CD19, CD20 and CD79a/b have been shown to be physically and functionally associated with MHC II and therefore were candidates. However, unlike the MHC II death response, CD19, CD20 and CD79a/b expression is believed to be B cell specific, indicating that these molecules are not likely to be involved in this response. MHC II is also known to associate with tetraspanins, including CD9 and CD37. However these molecules have very short cytoplasmic tails (8˜14aa) that lack defined signaling motifs. Therefore, it is believed that a novel MHC II associated protein(s) is involved in transduction of signals that mediate the death response. To explore this possibility the present inventors undertook proteomic analysis of MHC II associated proteins. Lysates of K46 cells were prepared using a mild detergent (CHAPS) that preserves weak protein-protein interactions, and then immunoprecipitated MHC II and associated proteins using anti-MHC II mAb beads. Proteins were eluted from the beads and identified using nanoLC-MS/MS. Forty-one proteins were recovered from the MHC II immunoprecipitates based on the detection of at least two unique peptides from each. MHC II α and β chains were identified by 6 and 14 peptides, respectively, confirming effectiveness of the affinity purification.

MHC II-associated proteins containing transmembrane domain(s) and cytoplasmic signaling motifs were targeted and searched. A “hypothetical protein”, RIKEN cDNA 2610307008, was implicated by three unique peptides predicted by its DNA sequence. Analysis showed that this hypothetical protein is predicted to contain four transmembrane domains by SOSHI (http://bp.nuap.nagoya-u.ac.jp/sosui/), TMHMM (http://www.cbs.dtu.dk/services/TMHMM-2.0/) and TMpred (http://www.ch.embnet.org/software/TMPRED_form.html) along with multiple signaling motifs by ELM (http://elm.eu.org/) and scan site (http://scansite.mit.edu) including an ITIM (immunoreceptor tyrosine-based inhibitory motif, SVY²⁴⁴EIL) (FIG. 1). The present inventors have designated this protein “MPYS” based on its N-terminal methionine-proline-tyrosine-serine amino acid sequence.

The mpys gene was cloned from a cDNA library produced from the murine K46 B lymphoma. The gene encoded a 378 aa protein with predicted mass of 42 kDa (FIG. 1). The sequence does not show significant homology to known or predicted proteins, suggesting MPYS belongs to a novel, single member class of proteins. Human MPYS is ˜80% homologous with mouse and no invertebrate homologues of mpys were found in the database (FIG. 1).

To explore protein expression and function a polyclonal Ab was raised against the C-terminal 101 aa of murine MPYS. The polyclonal Ab was then used to immunoprecipitate endogenous MPYS from K46 cell lysates. Eluates were analyzed by nano-LC/MS/MS. Antibody reactivity with MPYS was confirmed by detection in eluates of 15 unique peptides covering over 50% of the total amino acid sequence, including peptides from the N- and C-termini of the protein. Consistent with MPYS association, 2 and 4 peptides derived from MHC II cc and 13 chains, respectively, were found in the immunoprecipitate. This association was confirmed by immunoblotting.

To evaluate the cell surface expression of MPYS, mpys-HA was expressed in K46 cells. These cells were designated KHA. Anti-HA immunoprecipitation (IP) of lysates of surface biotinylated K46 cells followed by SDS-PAGE, transfer and avidin blotting revealed that MPYS was biotinylated, and therefore was on the cell surface.

To confirm the predicted topology of MPYS protein, two flag tagged mpys constructs were synthesized. One had a flag tag inserted in the predicted large extracellular loop (LEL-flag). The other had a flag tag in the predicted small extracellular loop (SEL-flag). These two constructs were expressed in K46 cells and stained the cells with anti-Flag Ab. The flag tagged MPYS proteins were detected on cell surface. On the contrary, polyclonal Ab that is against the predicted cytoplasmic tail of MPYS did not stain intact cells.

To investigate MPYS distribution in cells, a MPYS-GFP fusion construct was prepared and expressed in K46 cells. Confocal microscopy showed that while some MPYS was found on the cell surface, a large proportion was actually localized to mitochondria.

The transmembrane region of MPYS contains four charged residues and two cysteines. Such residues are believed to often mediate inter- or intra-protein interactions. To determine if MPYS can form protein complexes, the membrane permeant chemical crosslinker DSP was used to form covalent bonds between neighboring proteins and performed SDS-PAGE analysis on K46 whole cell lysates on a non-reducing SDS-PAGE gel. In addition to the monomer, MPYS immunoblotting revealed an ˜80 kDa band, a band double the size of the MPYS monomer. This indicated that most MPYS exists as a dimer within cells. The blot was stripped and reblotted with MPYS Abs in the presence of a blocking peptide (the 101 aa used to generate the MPYS Ab). No protein bands were recognized, indicating that MPYS Ab blotting was specific. Sequential blotting for CD19 provided an additional control for equivalent loading.

To assess tissue distribution of MPYS, lysates of various B and T cells were SDS-PAGE probe fractionated and transferred. Anti-MPYS reacted with a predominant 40 kDa species in spleen and thymus. Splenocytes have higher MPYS expression than thymocytes consistent with higher level expression in B cells. MPYS was also present in dendritic cells.

To assess potential changes in MPYS expression during B cell development and differentiation, whole cell lysates from B lineage tumors were probed with MPYS Ab. MPYS Ab recognized a 40 kDa band that was enhanced in K46 cells expressing HA-tagged mpys gene. MPYS was highly expressed in cells representing mature stages of B cells (Bal17 line) but weakly expressed in pre-B cells (70Z/3 line), immature B cells (WEHI 231 line) and memory B cells stages (A20 line). It was not detected in plasma cells (J558L line). Thus it appears to be expressed throughout the B lineage prior to the plasma cell stage, but occurs at highest levels in mature B cells.

### MPYS Possesses Inhibitory Signaling Function

The cytoplasmic tail of MPYS contains ITIMs, motifs known to recruit the inhibitory signaling effectors SHP-1 and SHIP. To assess the ability of MPYS to serve an inhibitory function, K46 cells were stimulated with anti-MHC II mAb for 2 min before cells were lysed and MPYS was immunoprecipitated, fractionated by SDS-PAGE and subjected to immunoblotting analysis. Anti-phosphotyrosine blotting revealed that MPYS is tyrosine phosphorylated upon MHC II crosslinking. Reprobing the blot with anti-SHP-1 and SHIP Abs showed that phosphorylated MPYS bound SHP-1 and SHIP. Notably, a 32 kDa unknown tyrosine phosphorylated protein was also associated with MPYS. Thus it is believed that MPYS engages negative signaling effectors when tyrosine is phosphorylated, consistent with its immunoreceptor tyrosine-based inhibitor motif (ITIM).

Studies of various inhibitory receptors suggest that SHP-1 and SHIP recruitment results in inhibition of calcium mobilization. To assess whether MPYS mediates this function, MPYS was overexpressed in K46 cells. MPYS overexpression led to a significantly reduction in MHC II mediated calcium mobilization. These data indicate that MPYS act in feedback regulation of some MHC II signals, i.e., those that lead to calcium mobilization.

The present inventors have also found that cells overexpressing MPYS tend to be lost from populations during culture, suggesting MPYS may have a negative effect on cell growth. To further explore this possibility, MPYS-GFP fusion construct was expressed in A20 cells. Sorted MPYS-GFP positive A20 B lymphoma cells lost MPYS-GFP expression within 10 days. In contrast, expression of GFP alone in A20 was maintained indefinitely. These data indicate that MPYS functions as a negative regulator of cell growth/viability.

### Knock-Down of MPYS Expression in K46 Cells Inhibits MHC Class II Aggregation-Induced Cell Death and ERK Activation

To further study the role of MPYS in MHC II signaling, a short hairpin RNA (shRNA) targeting exon 5 (sh5) or exon7 (sh7) of the mpys gene was prepared and used to knock down MPYS expression in K46 cells. Cells expressing sh5-RNA displayed a >90% reduction in MPYS expression while in cells expressing sh7-RNA MPYS expression decreased >80%. Surface expression of IgM, MHC II, CD19, CD45, CD80 and CD86 was not altered by shRNA expression. Contrary to MPYS over-expression, MPYS knock-down increased the growth rate of K46 cells.

The role of MPYS in the death response was examined by assessing anti-MHC II induction of death in K46 cells expressing MPYS knock-down constructs. K46 cells expressing a control luciferase shRNA (luc) or MPYS sh5 knock-down shRNA (sh5) were stimulated with biotinylated mAb M5/114 and avidin, and cell death was measured by Annexin V/PI dual staining. Biotinylated mAb 2.4G2 was used as an isotype control. MHC II mAb-induced cell death was reduced significantly in K46 cells expressing the sh5MPYS construct (where MPYS expression is diminished by >90%). An effect of MPYS knock-down was noted at all doses of anti-MHC II mAb used. Similar results were observed in K46 cells expressing sh7MPYS knock-down construct. Similar results were observed when other anti-MHC II antibodies were used for stimulation. These data indicate that MPYS expression is essential for anti-MHC II mAb induction of B cell death.

The effect of MPYS knock down on ERK activation was also investigated. Lysates of Luc or sh5 shRNA expressing K46 cells were probed fractionated and transferred using phospho-ERK Ab. MPYS knock-down inhibited MHC II mAb-induced ERK activation. These findings indicate that MHC class II signaling of cell death is dependent on MPYS-linked ERK activation.

Based on the experimental results, it appears that the death response is mediated by src-family kinase independent activation of MPYS and downstream ERK. Further, PP2 sensitive anti-class II activated signaling events, including calcium mobilization, and MPYS tyrosine phosphorylation and association with SHIP-1 and SHP-1, do not appear to be required for the death response.

To determine whether the MPYS dependence of ERK activation reflects a requirement that MPYS interact directly with ERK, cells were activated with MHC II mAb, before being lysed and subjected to MPYS immunoprecipitation. Immunoprecipitates were analyzed by SDS-PAGE and ERK immunoblotting. Under conditions in which SHP-1 recruitment to MPYS was observed, no noticable recruitment of ERK was detected.

MHC II independent aggregation of MPYS leads to cell death signaling. To test the ability of MPYS to transduce death signals when aggregated independently, K46 cells expressing flag tagged MPYS (LEL-MPYS) were stimulated with anti-flag and measured death by propidium iodide staining. Anti-flag treatment led to a ten fold increase in PI staining cells within 15 hours of stimulation. This response, while significant and antibody dose dependent, was somewhat less than that induced by anti-MHC II. These experiments show that MPYS functions as a transducer of death signals, and thus mediate death signaling in MHC II negative cells, such as T cells.

### Discussion

The heterodimeric MHC class II protein complex mediates binding of antigenic peptides and presentation of these peptides to CD4+ T cells for their consequent activation, proliferation, and differentiation. Under some circumstances it is important that cells presenting antigen be eliminated once they have served their function. It is believed that this may occur by TCR-induced MHC class II-mediated induction of apoptotic death. Such a mechanism would ensure elimination of only APC that had presented specific, potentially offensive antigens, and function to terminate responses to those antigens. Although not surprising for dendritic cells, it seems somewhat counterintuitive that such a mechanism would eliminate B cell APC, some of whose daughters would be expected to become antibody secreting cells. It is believed that some ex vivo B cells die an apoptotic death following aggregation of their MHC class II. This fate may be reserved for a certain B cell subpopulation whose continued antigen presentation and/or antibody production might be disadvantageous for the animal. For example, this mechanism may be important in elimination of autoreactive B cells.

Nano-LC/MS/MS analysis of MHC class II associated proteins revealed a previously unknown membrane tetraspanner that is associated with class II and expressed in both mature B cells and dendritic cells; cells that undergo apoptotic death in response class II aggregation. Consistent with transmembrane signaling function, the protein, termed MPYS, is found on the cell surface and contains multiple sites of predicted protein-protein interaction in its 140aa cytoplasmic tail. Based on these features the role of MPYS in apoptotic signaling was examined. MPYS was tyrosine phosphorylated following MHC class II aggregation. Consistent with its content of ITIMS, this phosphorylation was associated with its binding SHP-1 and SHIP-1. The Src inhibitor PP2 blocked MPYS tyrosine phosphorylation and recruitment of SHP-1 and SHIP-1. This shows that inhibitory signaling by these molecules is operative during class II signaling. MPYS overexpression inhibited class II calcium signaling and placed cells at a competitive disadvantage in terms of growth. Conversely, MPYS knock down promoted growth. Taken together these data show that at least two signaling pathways emanate from MHC class II on these cells. It is believed that one of them acts through Ig-α/β and SRC-family kinase activation to mediate calcium mobilization. A second, believed to be acting through MPYS tyrosine phosphorylation and recruitment of SHP-1 and/or SHIP-1, mediates inhibition of calcium signaling and cell growth.

MPYS expression was found to be required for MHC class II transduction of signals leading to ERK activation and apoptotic death. These responses were found to be linked since the ERK inhibitor PD98059 blocked both ERK phosphorylation and the cell death response. The class II death response was not affected by SB203580 or Ly294002, blockers of the Akt and p38 responses that also accompany class II signaling. Surprisingly however, neither MHC class II-mediated activation of ERK nor apoptosis was inhibited by the src-family tyrosine kinase inhibitor PP2. These findings indicate that a third signaling pathway emanates from MHC class II. This pathway involves tyrosine phosphorylation independent, but MPYS dependent, ERK activation and leads to cell death. It can be activated by “direct” antibody-mediated aggregation of MPYS, indicating that this signaling pathway is autonomous.

### Example 2

### MPYS/MPHS and Susceptibility to Infectious Disease

### Immunoprecipitation, Immunoblotting and Cell Surface Biotinylation

Transient transfection, coimmunoprecipitation, Western blot analysis and cell surface biotinylation were performed as described above.

### Reagents

Recombinant poly(I:C) (Invivogen), LPS (Sigma), poly (dA:dT) (Sigma, P0883), FLAG mAb M2 (Sigma, F-3165)), HA mAb 16B12 (Covance, AFC-101P), 4G10 phospho-tyrosine mAb, actin Ab (I-19) (Santa Cruz, sc1616), Tyk2 mAb (Transduction Lab, T20220), JAK1 rabbit polyclonal Ab (Upstate, 06272), JAK2 polyclonal Ab (Millipore, AB 3804), STAT1 polyclonal Ab (Santa Cruz, sc-346). MPYS rabbit polyclonal Ab, the 293-TLR4/MD2/CD14, 293 cells and 293-TLR3 cells have been described by Xu et al., in Mol. Cell. 19, 727-740.

### Constructs

NF-κB luciferase reporter, ISRE, IFNβ, IFNγ, AP-1, p53, IL-2, NF-AT, cyclin A, ELK, c-Jun and CHOP luciferase reporter constructs were described before. Id. Mammalian expression plasmids for TBK1, Myd88, Trif, IRF3, IRF7, IRF3-dominant negative mutant, IKKE, RIG-I, Tram, VISA and their mutants were described. Id. Mammalian expression plasmids for mouse and human MPYS mutants were constructed by standard molecular biology techniques.

### Reporter Gene Assays

293 GT cells and their derivatives (˜1×10⁵) were seeded in 24-well dishes and transfected the following day using Effectene transfection reagent kit (Qiagen, cat #301427). Reporter assays were performed as previous described. Id.

### Results

Human MPYS (i.e., MPHS) potently activates the IFN-stimulated response element (ISRE) but only weakly for the IFNβ promoter in the 293GT cells

The human MPYS (i.e., MPHS) cDNA and various promoter-luciferase constructs were co-infected into the 293GT cells. 293GT cells do not have detectable endogenous MPYS expression. It was discovered that MPYS expression in the 293GT cells led to potent ISRE activation (−90 folds) but a relatively weak IFNβ promoter and NFκB activation (<3 folds). Over-expressed Traf2 induced potent NF-ηB activation in 293GT cells was also observed. In contrast, VISA expression in 293GT cells potently activated IFNη promoter (>200 folds). Both, VISA and MPYS induced IFNβ activations were inhibited by a dominant negative IRF3 mutant.

No activation was observed for AP-1, IL-2, c-Jun, p38, NFAT, IFNγ, p53, and cyclin A by MPYS in 293GT cells. MPYS weakly activated ERK.

Murine MPYS was dimerized in mouse B cells. To see if human MPYS (i.e., MPHS) also forms dimer when expressed in 293GT cells, a non-reducing gel was run and detected MPYS with the above described polyclonal anti-MPYS Ab. Most of expressed human MPYS was seen at a size of ˜80 kDa which is roughly the size of a MPYS dimer. To further demonstrate that MPHS also exists as a dimer, HA-MPHS and Flag-MPHS were co-expressed in the 293GT cells. Indeed, Flag-MPHS was associated with HA-MPHS. As a control, Flag-MPHS did not bind to HA-Igβ. This indicated that MPHS exists primarily as a dimer when expressed in 293GT cells.

As discussed above, MPYS can be tyrosine phosphorylated and recruit SH2 domain-containing proteins. MPHS was also tyrosine phosphorylated when expressed in 293GT cells. Moreover, phosphorylated MPHS recruited STAT1. There are five conserved tyrosine residues between human and mouse MPYS. It is believed that three of them, Y244, Y260, Y313, are likely able to bind SH2 domain. Mutating each one of them led to decreased ISRE activation by MPHS. These mutations did not appear to affect protein expression.

Though activated IFNβ can lead to ISRE activation through the IFNβ receptors mediated JAK-STAT signaling pathway, the discrepancy between the potent ISRE and a relatively weak IFNβ promoter activation appears to suggest that the ISRE activation induced by MPHS may not be completely due to the weak IFNβ activation. Without being bound to any theory, it is believed that to efficiently activate IFNβ promoter, three transcriptional factors, NFκB, ATF/c-Jun and IRF3/IRF7 have to act synergistically. MPHS did not appear to activate c-Jun, and relatively weakly activated NFκB. Thus, it is believed that by associating with STAT1, MPHS directly activates the JAK-STAT pathway.

Tyk2 is Associated with MPYS and Required for MPYS Induced ISRE Activation

Type I IFN receptors are associated with Tyk2 and JAK1. MPHS contains a box 1 motif (⁸PxxPxP) in its N-terminal membrane proximal region. It is known that IFNs and other cytokine receptors use the juxtamembrane box 1 motif to recruit JAKs. Mutating the box 1 motif in mouse MPYS (⁸PxxPxP to ⁸PxxAxA mutant) led to no ISRE activation by MPYS. Furthermore, mutated box 1 motif in mouse MPYS acted as a dominant negative mutant. The tail-less MPYS mutant was also a dominant negative mutant. Western blot showed that the PxP mutant has substantially the same expression as the wt MPYS.

It was found that MPYS specifically bind Tyk2, but not JAK1 and JAK2. Using siRNA against Tyk2, endogenous Tyk2 expression was knocked down in 293 cells. Knock down Tyk2 expression decreased MPHS induced ISRE activation. These data show that MPYS, by association with Tyk2-STAT1, can directly activate the ISRE signaling.

The present inventors have observed that though MPHS relatively weakly activates IFNβ promoter in 293GT cells (<3 folds), it strongly activated IFNβ promoter in the TLR4-293GT cells (>50 folds). Interestingly, the NFκB activation was still very weak in the TLR4-293T cells (<3 folds) and there was no significant AP-1 activation. Notably, MPHS only weakly induced IFNβ production in the TLR3-293GT cells (−10 folds) though it did enhance poly(I:C) induced IFNβ activation in the TLR3-293GT cells.

Comparing the ISRE and IFNβ activation in the 293GT and TLR4-293GT cells, it was found that MPHS induced ISRE activity was about the same in both cells. However IFNβ activation induced by MPHS was much stronger in TLR4-293GT cells than in the parental 293GT cells.

The TLR4-293GT cells responded strongly to transfected poly(I:C). Because the TLR4-293GT cells don't have endogenous MPYS, this shows that MPHS is not essential for cytosolic dsRNA induced IFNβ response though MPHS expression did enhance its response. Surprisingly and unexpectedly, unlike the transfected Poly(I:C) and B-DNA, which increased MPHS induced IFNβ activation, it was found that LPS stimulation reduced MPHS induced IFNβ activation. However, LPS did not appear to reduce MPHS induced NFκB activation. This shows that MPHS induced IFNβ pathway may overlap with LPS induced IFNβ pathway. In fact, it was observed that a small percentage of expressed MPHS was associated with TLR4 in the TLR4-293GT cells.

It has been shown that B-DNA induced IFNβ response was significantly decreased in MPYS deficient MEF cells. Both the 293GT and TLR4-293GT cells are negative for the endogenous MPYS expression. It was found that the 293GT cells were relatively unresponsive to B-DNA activation. Expression of MPYS did not change responsiveness to B-DNA activation. However, B-DNA did activate IFNβ in the TLR4-293GT cells. Expressing the dominant negative MPYS did not appear to inhibit B-DNA induced IFNβ. These results show that MPYS is not necessarily required for B-DNA response.

TBK1 phosphorylates transcription factors IRF3 and plays an important role in activating IFNβ. It has been shown that MPYS induced IFNβ activation requires TBK1 expression. A dominant negative IRF3 mutant ablated MPYS induced IFNβ activation. Thus it appears that MPYS uses the TBK1/IRF3 pathway to activate IFNβ.

It has also been found that MPYS strongly co-immunoprecipitates with over-expressed TBK1 but only weakly co-immunoprecipitates with another ser/thr kinase IKKE. It was observed that the molecular weight of MPYS increased in cells over-expressing TBK1 and IKKE. Since TBK1 and IKKE are kinases, this observation shows that MPYS appears to be phosphorylated by TBK1. In fact, X-protein phosphatase treatment eliminated the additional MW on MPHS. Co-expressing MPYS and TBK1 also showed synergy in activating of ISRE as well as IFNβ. Thus, it appears that MPYS activates the TBK1/IRF3 pathway.

The cytoplasmic tail of MPYS contains 14 Ser and 6 Thr which are conserved between human and mouse. To explore whether these residues were important for MPYS induced ISRE activation, two of the conserved Ser (S240 and S242) were mutated into alanines. It was found that the S242A mutation had a deteriorate effect on MPYS induced ISRE activation. Thus S242 could be a phosphorylation site for TBK1.

The present inventors have discovered that MPYS is important for the anti-viral response. Furthermore, it was shown that MPYS interacts with VISA (also called IPS-1, Card-IF, MAVS). It is known that mitochondria protein VISA plays an essential role in RNA virus induced anti-viral response. Both MPYS and VISA appear to use the TBK1/IRF3 pathway to activate IFNβ. MPYS interacts with over-expressed VISA though not as strong as TBK1. At best, RIG-I, the cytosolic receptor for dsRNA, appeared to only very weakly interact with MPYS.

Both VISA and MPYS are transmembrane proteins and found in the mitochondria. Using a series of truncated VISA mutants, it was determined that the C-terminal 360˜540aa region of VISA were sufficient for MPYS interaction. This region contains the transmembrane domain of VISA. Thus, it appeared that VISA interacts with MPYS through their transmembrane regions.

VISA can activate IFNβ in the absence of MPYS in 295GT cells. Transfected Poly(I:C) can also activate IFNβ independent of MPYS in the TLR4-293GT cells. The present inventors have also found that the expression of VISA strongly activated IFNβ in TLR4-293GT cells in the absence of MPYS. However, the expression of MPYS enhanced VISA induced IFNβ. The dominant negative MPYS mutant (tailless MPYS) decreased VISA induced IFNβ activation. However, knock down endogenous VISA expression in the 293GT cells did not affect MPYS induced IFNβ activation.

The non-Synonymous SNPs in Human MPYS have Altered IFNβ and ISRE Stimulating Activities

MPYS plays a critical role in host innate immune response. Thus, the non-synonymous SNPs of MPYS found in human populations have clinical importance. Currently, there are three validated non-synonymous SNPs in human MPYS. See Table 1.

TABLE 1

| SNPs | dbSNP # | Population Frequency |
|---|---|---|
| R71H | rs11554776 | Asian: 0.146; European 0.017 |
| R232H | rs1131769 | Asian: 0.01; European 0.01 |
| R293Q | rs7380824 | Asian: 0.146; European 0.017 |

They were found with different population frequencies. When expressed in the 293GT cells, the R71H and R293Q decreased ISRE activity while the R232H had a near normal ISRE activation. When expressed in the TLR4-293GT cells, the R71H and R293Q decreased IFNβ activation while the R232H had increased IFNβ activity. All three SNPs had a similar expression level compared to the wild type human MPYS. These SNP can be useful diagnostic biomarkers for susceptibility to infectious diseases (e.g., vesicular stomatitis virus, herpes simplex virus 1 infection and Sepsis response) and lung diseases (COPD or squamous cell lung carcinoma and lung adenocarcinoma) in human.

### Example 3

### Thiol Reductase Activity of MPYS

### Cell Culture

RAW264.7, K46 B lymphoma cells and the 293GT cells were maintained in IMDM supplemented with 5% FBS. 293MT cells were maintained in DMEM (GIBCO, cat: 11965), 5% FBS (Biosource, 200p-500HI), sodium pyruvate (GIBCO 11360, 1 mM), HEPES buffer (GIBCO 15630-080, 10 mM) and 2-ME (50 μM) (Life Technologies, Gaithersburg Md.). The RAW264.7 cells stably expressing the IFNβ-Luciferase construct were generated by co-transfecting RAW264.7 cells with pGL3-IFNβ-Luciferase (Promega) and p-Puromycin plasmids (Clonetch). The stable cell line was established under puromycin selection (4 μg/ml).

### Reporter Gene Assay

Cells were seeded in 24-well dishes (˜1×10⁵/ml) and transfected the following day using Effectene transfection reagent kit (Qiagen, 301427). Reporter assays were performed as previous described by Bin et al., in J Biol. Chem., 2003, 278, 24526-24532. All experiments were repeated at least three times and results are representative.

### Thiol Reductase Assay

Thiol reductase assay was done using the thioredoxin reductase kit (Sigma, CS0170). Briefly, 293MT cells expressing MPYS were lysed in CelLytic M buffer (Sigma, C2978) with protease inhibitors (Roche, 11836153001). Proteins in the WCL were quantified using Biorad DC Protein assay kit (500-0016). The assay was done in 96-well plates containing 0.2 mg/ml NADPH and 1.188 mg/ml DTNB. The plates were read in a VMAX Microplate reader using Softmax pro4.7.1 at 10 second intervals for 2 min at 412 nm. The WCL was then added in the well and the plate was read for another 2 min. Thiol reductase activity was calculated as ΔA412/min.

### Oxidoreductase Assay

Oxidoreductase activity was assayed using rabbit anti-MPYS or anti-CD22 polyclonal Ab-conjugated Sepharose bead immunoprecipitates from 50×106 cells lysates (1% n-dodecyl-β-D-maltoside (DM), 50 mM NaH₂PO₄ pH 8.0, 300 mM NaCl) on ice for 1 hr. Beads bound immunoprecipitates were washed twice in the reaction buffer (50 mM NaH₂PO₄, pH 7, 0.1% DM, 300 mM NaCl, 0.5 mM EDTA) and then incubated in the same buffer containing 25 μM DTT at 37° C. for 10 min. Substrate, biotinylated-murine IgG2a (D3.137) (10 μM) was denatured by incubation in 0.1% SDS, 0.05% Triton buffer at 65° C. for 30 min. The denatured biotin-IgG2a (2 μl) was added to the 50 μl beads in reaction buffer and this mixture was incubated at 37° C. for 1 hr. Clarified supernatant was fractionated by non-reducing SDS-PAGE electrophoresis and transferred to PVDF membrane. Biotinylated IgG2a and component immunoglobulin light and heavy chains were detected by streptavidin-Alexa Fluor 680 and visualized using an ODYSSEY Infrared Imaging System (LI-COR).

### Immunoprecipitation

Cells were lysed in 0.33% CHAPS buffer (150 mM NaCl, 10 mM Tris pH 7.5, mM sodium pyrophosphate, 2 mM Na₃VO₄, 10 mM NaF, 0.4 mM EDTA, 1 mM PMSF, and 1 μg/ml each of aprotinin, α1-antitrypsin, and leupeptin) at 4° C. for 1 h. Cell lysates were centrifuged at 12,000 g at 4° C. for 10 min. Immunoprecipitation was done in the lysates with indicated Ab-conjugated Sepharose beads.

### Measurement of Intracellular ROS

Cells were washed and suspended in PBS, and then culture with H2DCFDA (5 μM) (Invitrogen, D399) for 20 min at 37° C. They were then washed in PBS and the fluorescence was measured by flow cytometry.

### RT-PCR

Human IFNβ (5′-CAGCAATTTTCAGTGTCAGAAGC-3′ and 5′-TCATCCTGTCCTTGAGGCAGT-3′) and HPRT1 (5′-GGACAGGACTGAAAGACTTGCTCG-3′ and 5′-TCCAACAAAGTCTGGCCTGTATCC-3′) were amplified using PrimeSTAR DNA polymerase (TaKaRa, R010A) with the following program: 94° C. 30 sec, 30 cycles of 98° C. 10 sec, 58° C. 5 sec and 72° C. 30 sec, then 72° C. for 5 min.

Listeria monocytogenes Infection

RAW264.7 cells expressing the IFNβ-luc construct were infected with Listeria monocytogenes strain 10403S at multiplicity of five bacteria/cell. Cells were washed at 1 h after infection. Live bacterial were killed by addition of gentamicin. Whole cell lysates were prepared 5.5 h after infection. Luciferase activity was read with BD Monolight kit and Synergy reader.

### Results

Reactive Oxygen Species (ROS), including the superoxide anion, hydrogen peroxide and hydroxyl radicals, are typically generated from the incomplete reduction of oxygen. Most intracellular ROS are generated in mitochondria due to electron leakage along the respiratory chain. ROS are also generated in the endoplasmic reticulum (ER) during the unfolded protein response (UPR). Bacterial infections induce transient production of large amounts of ROS, a process called the oxidative burst, on the membrane of endosomes within phagocytes such as neutrophils and macrophages.

Potent ROS mediated oxidative stress causes irreversible cell damage and eventually cell death. More modest elevation of ROS activates “redoxin signaling” which uses cysteine residues as redox sensors to mediate inflammatory responses. Cys can be reversibly oxidized to sulfenic acids, S-glutathionylated or S-nitrosylated cysteines, or disulfide bonds. S-glutathionylations of IRF3 and Cys-179 of the IKK-β subunit inhibit their activation. Oxidation of catalytic Cys in caspases and protein tyrosine phosphatases (PTP) inhibits their activation. Thus, ROS-mediated post-translational modifications on Cys regulate the biological activities of many proteins. High cellular ROS levels have been linked to ageing, human cancers, inflammatory, lung and cardiovascular diseases. Antioxidants show protective effects for certain cancers and cardiovascular diseases.

MPYS is a potent IFNβ stimulator that plays a role in innate immune responses of fibroblasts to RNA and DNA viruses. MPYS contains four predicted α-helical transmembrane domains (TM) with a potential redox-active Cys₈₈-L-G-Cys₉₁ (C₈₈xxC₉₁) motif at the N-terminus of the second TM. The CxxC motifs are often found in the oxidoreductases and are at least partly responsible for their catalysis of redox reactions.

In CxxC redox motifs, it is believed that the low thiol pKa value of the first Cys (Cys1) is at least in part responsible for the oxidoreductase activity. The electrostatic effect of the positive charged Arg-86 in the −2 position of the Cys1 (Cys-88 of MPYS) may lower its thiol pKa. Its position at the N-terminus of an α-helice (the second TM), with its positive NH microdipoles, may also lower the pKa of Cys1. The two central residues in the CxxC motif, because of their proximity, can also affect the pKa of Cys1. In fact, oxidant oxidoreductases usually have a positively charged His between the two Cys while reductant oxidoreductases have hydrophobic residues in this position. Thus, the C₈₈LGC₉₁ motif of MPYS indicates that it is a reductant oxidoreductase.

The combination in MPYS of a CxxC motif with a four-helical bundle structure is shared by a group of structurally unique oxidoreductases that include the ER oxidoreductases, Ero1, the bacterial periplasm oxidoreductases, DsbB, the mitochondrial intermembrane space (IMS) oxidoreductases, Erv1, the vaccinia virus E10R protein and the E10R homologs found in all cytoplasmic DNA viruses. It should be noted that Ero1, Erv1, E10R and DsbB share structural but not sequence similarity.

The present inventors have discovered that MPYS is a thiol reductase and that its oxidoreductase activity is at least in part responsible for its stimulation of IFNβ production. It was also discovered that high levels of ROS cause MPYS oxidation and loss of its ability to activate IFNβ expression. Thus MPYS is a transducer whose signaling function requires reductase activity, and this activity is regulated by oxidative stress.

### Results

To test MPYS's thiol reductase activity, human MPYS was expressed in the 293MT line, a derivative of HEK293T cells, and confirmed that it activated IFNβ and NFκB promoters. 293MT was employed because it expresses no detectable endogenous MPYS, and thus provides a null control.

The effect of MPYS on total cellular reductase levels was then assessed. Whole cell lysates were prepared from 293MT cells transduced with MPYS or empty vector (Vec), and examined their ability to reduce 5,5′-dithiobis(2-nitrobenzoic) acid (DTNB) in the presence of NADPH. Reduction of DTNB gives rise to 5-thio-2-nitrobenzoic acid (TNB), which absorbs at 412 nm. It was found that expression of MPYS in the 293MT cells increased the rate of TNB production.

Endogenous MPYS was immunoprecipitated from murine B lymphoma cells (K46), pre-activated by reduction with 25 μM DTT, then incubated with biotinylated IgG substrate. After the reaction, the protein mixtures were analyzed by non-reducing SDS-PAGE and probed with streptavidin to detect the biotinylated IgG. MPYS, but not a control CD22 IP, reduced IgG (H2L2) generating free immunoglobulin heavy (HC) and light (LC) chains.

Non-reducing SDS-PAGE analysis revealed that MPYS itself changed from a mostly reduced form to a mostly oxidized form during the reaction. Unlike GILT, MPYS activity was detected at pH 7, but not at pH 5. These data showed that the IFNβ stimulator, MPYS, is a thiol reductase.

To assess the role of the C₈₈xxC₉₁ redox motif in MPYS thiol reductase activity, the cysteines in the C₈₈xxC₉₁ motif were mutated to alanine and the effects of the mutation on activity were assessed. It was found that the thiol reductase activity decreased significantly when either residue was mutated. This was particularly true for the first Cys (C₈₈A). The C₉₁A mutant retained some ability to break the disulfide bonds in IgG. The expression levels of the mutants were similar to that of the WT MPYS.

Cys-147 is also conserved, lies close to a positive charged residue (Lys-149) and sits at the N-terminus of a α-helice (the 4th TM). As a consequence, Cys-147 may have a low pKa, and could also play a role in thiol reductase activity. It was found that the C₁₄₇A mutant, like the C₈₈xxC₉₁ mutant, lost reductase activity. Both the C₈₈xxC₉₁ and C147A mutants are expressed at the same level as the WT MPYS. Therefore, in addition to the C₈₈xxC₉₁ redoxin motif, Cys 147 appears to be required for MPYS thiol reductase activity and may, together with the C₈₈xxC₉₁ motif, form the active enzymatic site.

It was also noticed that after the thiol reductase reaction, a large portion of MPYS occurs in a ˜80 kDa SDS resistant form consistent with the predicted size of a homodimer. The present inventors have shown that MPYS is dimeric in mouse B cells. To determine whether MPYS occurs as a non-disulfide-bonded dimer that becomes intrachain disulfide bonded upon oxidation, the chemical crosslinker Dithiobis[succinimidyl propionate] (DSP) was used to detect the endogenous MPYS complex in mouse splenocytes. In non-reducing SDS-PAGE, DSP treated MPYS ran as a single band of ˜80 kDa, the predicted size of a MPYS homodimer while non-DSP treated mock (or DTT treated) MPYS ran at the monomer size (˜40 kDa). It was also discovered that Flag-tagged MPYS associates with HA-MPYS when the two were co-expressed. Thus, the ˜80 kDa band seen on the non-reducing gels is a MPYS homodimer. Since this homodimer is sensitive to SDS, its dimerization is not mediated by intermolecular disulfide bonds.

The appearance of the SDS resistant MPYS homodimer after the thiol reductase reaction indicated the formation of intermolecular disulfide bonds in homodimeric MPYS is a consequence of oxidation. Indeed the same SDS resistant MPYS homodimer was detected in splenocytes after H₂O₂ treatment. K46 mouse B lymphoma cells and RAW264.7 mouse macrophage cells also formed the MPYS homodimer with intermolecular disulfides (referred as oxidized MPYS) (FIG. 3) after H₂O₂ treatment. The mitochondrial electron transport chain complex I specific inhibitor, rotenone, also induced MPYS oxidation. Thus, both oxidative stress (e.g. H₂O₂) and the thiol reductase reaction can lead to formation of intermolecular disulfide bonds in homodimeric MPYS.

It was believed that high levels of ROS might affect MPYS thiol reductase activity by driving oxidation of Cys residues. Consistent with this hypothesis it was found that when MPYS was expressed in a different HEK293T derived line (293GT) that is spontaneously high in ROS, a significant portion of MPYS occurs as disulfide bonded dimers (oxidized MPYS). Finally, unlike MPYS in the 293MT cells, MPYS in the 293GT cells lost its ability to reduce the disulfide bonds in IgG. Thus the formation of the oxidized MPYS homodimer eliminates MPYS' thiol reductase activity and ROS, by virtue of oxidizing MPYS cysteines, disrupts its thiol reductase activity.

In addition to its oxidation and loss of thiol reductase activity, the present inventors have discovered that in 293GT cells MPYS was unable to stimulate IFNβ reporter activation. It is believed that MPYS requires its thiol reductase activity to mediate signaling of this response and therefore such signaling would be also sensitive to ROS inhibition. Rotenone is a specific inhibitor of mitochondrial electron transport chain complex I. TLR4-293T cells treated with rotenone exhibited increased cellular ROS level and oxidized MPYS. Moreover, rotenone completely inhibited MPYS induced IFNβ and NFκB reporter activation in the TLR4-293T cells.

Whether MPYS oxidation compromises its ability to transduce signals in response to a physiologic stimulus was examined. MPYS is required for Listeria monocytogenes induced IFNβ response in fibroblasts. It was found that knock down of MPYS in the RAW264.7 macrophage cells decreased Listeria monocytogenes induction of IFNβ responses. This showed that MPYS mediated Listeria monocytogenes induced IFNβ response in macrophage cells. Whether ROS could inhibit Listeria monocytogenes induced IFNβ response was also examined. Rotenone treatment induced MPYS oxidation. Moreover, rotenone treatment significantly inhibited Listeria monocytogenes induction of IFNβ production in these RAW264.7 cells. These data showed that by inhibiting the thiol reductase activity of MPYS, ROS inhibits Listeria monocytogenes activation of the IFNβ response.

C₈₈xxC₉₁ and Cys-147 are important for the thiol reductase activity of MPYS. It is believed that they would also be required for the IFNβ stimulation. It was found that both the C147A and C₈₈xxC₉₁ mutants significantly decreased IFNβ responses compared to wild type MPYS. Mutation of each Cys in the C₈₈xxC₉₁ motif (C88A and C91A) both inhibited the IFNβ response. This data shows that Cys-88, Cys-91 and Cys-147, which are essential for the thiol reductase activity of MPYS, are important for the activation of IFNβ by MPYS.

Dependence of MPYS IFNβ production on its reductase activity was surprising and unexpected in view of the fact that this response has been shown to require its ˜200aa-long tail which lies on the opposite membrane face from the redox motif. To address the interplay between these domains, a flag tagged tailless mutant of MPYS (TL-MPYS, truncated at aa182) was made and determined whether the cytoplasmic tail is also required for the thiol reductase activity. It was found that cells expressing the TL-MPYS had a similar thiol reductase activity as that of WT. Furthermore TL-MPYS, like WT MPYS, can reduce the disulfide bonds in the IgG. This indicated that the cytoplasmic tail and thiol reductase activities of MPYS function in parallel and complementary pathways to stimulate IFNβ production.

These data show that the thiol reductase activity of MPYS is required for its induction of IFNβ production. IFNβ stimulation requires the activation of both NFκB and IRF3. It was found that both the C₈₈xxC₉₁ and C147A mutants could not activate IRF3. Consistent with the observation that the C₈₈A mutant has the most significant effect on MPYS thiol reductase activity, the C₈₈A mutant had the most severe defect in IRF3 activation.

The C₉₁A mutant activated IRF3 normally. However, the C₉₁A mutant was defective in NFκB activation, which may explain its decreased IFNβ stimulation. The C₉₁A mutant showed some residual thiol reductase activity. While it is possible that the residue thiol reductase activity in the C₉₁A mutant is sufficient for IRF3 but not NFκB activation, it appears that the thiol reductase activity of MPYS is required for its activation of IRF3 and NFκB.

The ability of B-DNA to stimulate MPYS thiol reductase activity was examined. It was found that introducing B-DNA into the RAW264.7 cells resulted in MPYS oxidation, an indication of MPYS thiol reductase activation. Oxidized MPYS could be detected as early as 4 hrs following transfection (indicated by arrow). Oxidative stress of RAW264.7 using either Rotenone or H₂O₂ treatment also led to the formation of oxidized MPYS. However, it was found that B-DNA treatment only weakly increased cellular ROS level compared to LPS. LPS treatment of RAW264.7 cells induced oxidized MPYS at a later time point compared to the B-DNA treatment. The induction of MPYS oxidation was specific because Poly(I:C) treatment did not generate oxidized MPYS. Also, Poly(I:C), LPS and B-DNA, at the concentration used in above experiments, induced similar IFNβ production in the RAW264.7 cells. Knockdown of MPYS decreased B-DNA induction of IFNβ production. These data indicate that B-DNA stimulates MPYS thiol reductase activity.

### Discussion

MPYS, a potent stimulator IFNβ production, is a thiol reductase. MPYS thiol reductase activity is required for its stimulation of IFNβ production, and that the thiol reductase “signal” is transduced via NFκB and IRF3. This signal acts together with a signal emanating from the MPYS cytoplasmic tail in the induction IFNβ. Both the thiol reductase activity and IFNβ stimulation by MPYS can be inhibited by high levels of ROS. ROS stimulate MPYS oxidation as manifest by formation of intermolecular disulfide bonds within MPYS homodimers. It is believed that MPYS senses the redox state of the cell and under conditions of high oxidative stress is “turned off”, presumably by oxidation. It is also believed that MPYS mediates the ROS regulation of IFNβ response during innate immune responses to dsDNA and other virus.

ROS play an important role in regulation of innate immune responses and the strength of the ROS signals generally determines the outcome of such responses. For example, low levels of H₂O₂ activate the p53 antioxidant response, whereas high levels trigger p53-dependent apoptosis. The effect of ROS on MPYS function also appears to depend on the levels of ROS. It was found that the B-DNA response, which induces low levels of ROS, activates MPYS thiol reductase activity. In addition, it was found that rotenone treatment, which produces high levels of cellular ROS, inhibits the MPYS-mediated IFNβ response. In some instances, it is believed that high levels of ROS cause irreversible modification of the Cys in MPYS, which eventually lead to MPYS mediated cell death. On other instances, low levels of ROS generate reversible Cys modification on MPYS and lead to activation of its thiol reductase activity and thus the IFNβ mediated anti-viral response. NLRX1, a negative regulator of the anti-viral response, triggers strong ROS production (comparable to the level triggered by TNFa). Both NLRX1 and MPYS can be found in mitochondria. Thus, it is possible that NLRX1 negatively regulates the IFNβ response by producing strong ROS thereby inhibiting MPYS thiol reductase activity.

Oxidoreductases are found in ER, e.g. Ero1, and intermembrane space (IMS) of mitochondria, e.g. Erv1. Both ER and IMS originated from the periplasm of prokaryotes. Previous over-expression studies have placed MPYS in the ER and mitochondrial. The mitochondrial outer membrane is physically and physiologically connected to the ER. This physical link may facilitate Ca⁺² and it is believed that ROS “communicates” between ER and mitochondria. Mitofusin 2 had been identified as the protein that bridges ER and mitochondria. Mitofusin 2 associates with VISA (also known as MAVS, IPS-1 and CardIf) and is a negative regulator of the IFNβ response. MPYS also has been shown to bind to VISA. Therefore, it is likely that MPYS also associates with Mitofusin 2.

For MPYS that are present in the ER, the Cysteines in its ectodomains (Cys-88, Cys-91 and Cys-147) is believed to face the ER lumen. The present inventors have discovered that the ER stress inducer, Brefeldin A, also generated oxidized MPYS. Thus, it is believed that MPYS act in ER stress sensing.

The IMS is connected to the cytosol by porins in the outer membrane of mitochondria which allow the diffusion of small ions like glutathione. Thus the environment of IMS is less oxidizing than that of the ER lumen. As a result, MPYS in the IMS are believed to have a greater thiol reductase activity. Recently, a group of interacting mitochondrial proteins, including MPYS, VISA, NLRX1 and most recently Mitofusin 2, have been identified as key components of the innate intracellular viral sensing pathway. Without being bound by any theory, because the MPYS is a ROS sensor, it is believed that the antiviral response also utilizes mitochondrial ROS as second messenger.

Stimulation of IFNβ production has been shown to require activation of NFκB and IRF3, and the present inventors have shown that MPYS thiol reductase activity is required for IFNβ response to Listeria moncytogenes and B-DNA but not poly(i:c). The present inventors have also shown that MPYS-induced NFκB and IFNβ activation is inhibited by increased cellular ROS under conditions in which MPYS thiol reductase is lost. It is well established that NFκB is a redox-sensitive transcription factor. Oxidation of the Cys-62 of the NFκB p50 subunit has been shown to prevent DNA binding. H₂O₂ treatment inhibits TNF-α induced NFκB activation by oxidizing the Cys-179 of the IKK-β subunit. Recently, it has also been shown that the cysteines in IRF3 must be in a reduced state to activate IFNβ. Accordingly, it is believed that MPYS promotes IFNβ production by reducing regulatory cysteine residues in NFκB and IRF3.

It should be noted that the reductase activity alone is not sufficient for MPYS to activate IFNβ. The tailless MPYS has thiol reductase activity but can't stimulate the production of IFNβ. It is likely that MPYS needs its cytosolic tail to interface with other effector pathways important in signaling this response.

MPYS is required for B-DNA and Listeria monocytogenes-mediated IFNβ response in RAW264.7 macrophages. Thus MPYS deficiency is believed to lead to susceptibility to DNA virus or resistance to Listeria monocytogenes infection. However, VISA is not required for Listeria monocytogenes induced IFNβ response in the RAW264.7 cells suggesting that MPYS is not merely an adaptor protein for VISA.

MPYS has thiol reductase activity indicating it has pathological implications beyond infectious diseases. While MPYS participates in anti-MHC II mAb induced cell death, MPYS over-expression inhibits growth of mouse B lymphoma cells while knock-down of MPYS increases cell growth. Thus, it is believed that MPYS is a tumor suppressor. Indeed, MPYS mutations have recently been identified in human lung cancer patients. Lung pathologies are accompanied by elevated amounts of ROS production and MPYS is highly expressed in lung.

### Example 4

### Stimulation of ISRE by MPYS

### Materials and Methods

### Cell Culture

Wild type 293GT cells were maintained in DMDM (GIBCO, 11965 with D-glucose and L-glutamin), 10% Fetal Bovine Serum (Biosource, 200p-500HI), PenStrep (GIBCO, 15146, 100 UI/ml penicillin, 100 μg/ml streptomycin), gentamycin (50 μg/ml), sodium pyruvate (GIBCO 11360, 1 mM), HEPES buffer (GIBCO 15630-080, 10 mM) and 2-ME (50 μM) (Life Technologies, Gaithersburg Md.).

### Reporter Gene Assays

Procedure of Example 3 was followed.

### RT-PCR

Procedure of Example 3 was followed.

### Immunoprecipitation, Immunobloting and Cell Surface Biotinylation

Transient transfection, co-immunoprecipitation and immunoblotting were performed as previously described by the present inventors in Mol Cell Biol., 2008, 28, 5014-5026.

### Reagents

HA mAb 16B12 (Covance, AFC-101P), 4G10 phospho-tyrosine mAb, actin Ab (1-19) (Santa Cruz, sc1616), Tyk2 mAb (Transduction Lab, T20220), JAK1 rabbit polyclonal Ab (Upstate, 06272), JAK2 polyclonal Ab (Millipore, AB 3804), STAT1 polyclonal Ab (Santa Cruz, sc-346).

Tyk2 siRNA Knockdown

The siGENOME SMARTool siRNA for human Tyk2 (M-003182-02-0005) and siGENOME Non-Targeting siRNA Pool #2 (D-001206-14-05) were purchased from Dharmacon. Cells were transfected using DharmaFECT 1Transfection Reagent (T-2001-01) according to the manufacturer's protocol.

### Constructs

NF-κB, ISRE, IFNβ, IFNγ, AP-1, p53, IL-2, NF-AT, cyclin A, ELK, c-Jun and CHOP luciferase reporter constructs are described by Xu et al., in Mol. Cell., 2005, 19, 727-740. Mammalian expression vectors for mouse and human MPYS mutants were constructed by standard molecular biology techniques.

### Results

Human mpys cDNA and various promoter-luciferase constructs were co-transfected into 293GT cells. The human mpys cDNA employed was derived from a fetal liver library and represents the most common form of mpys found in human population (rs1131769). Expression of mpys led to a ˜90 fold activation in the ISRE reporter, but only weak (−3 fold), though reproducible, activation of IFNβ and NFκB promoters in the 293GT cells. To exclude the possibility that these cells lack the essential components for IFNβ activation, VISA was expressed in these cells and found that it stimulated an over 200 fold increase of IFNβ. These observations are consistent with an observation that ISRE activation by MPYS is not dependent on the IFNβ response. No noticeable endogenous MPYS expression was observed in this 293GT cell line.

MPYS activation of gene expression appears to be selective, since its over-expression did not appear to stimulate AP-1, IL-2, c-Jun, p38, NFAT, IFNγ, p53, and cyclin A reporters. However, weak activation of ERK by MPYS was observed consistent with the belief that MPYS mediates ERK activation leading to cell death. These findings indicate that MPYS activation of IFNβ and ISRE promoters is independent.

ISREs are activated by the Type I IFN αβ receptor through a JAK-STAT pathway. It is believed that the type I IFN αβ receptor associates with Tyk2 and JAK1 through a conserved juxtamembrane PxxPxP Box 1 motif and recruits STAT1/2 to its cytoplasmic phosphotyrosines. MPYS also contains a Box 1 motif (⁸PxxPxP) in its N-terminal membrane proximal region and multiple phosphotyrosine SH2 binding motifs in its C-terminal tail. Since MPYS occurs as a homodimer and has a LRR domain, its signaling of ISRE activation could resemble the Type I IFN αβreceptors. The Box 1 motif in MPYS (⁸PxxPxP to ⁸PxxAxA) was mutated and found that it abolishes MPYS' ability to activate the ISRE promoter. In fact, this Box 1 mutant acted as a dominant negative mutant consistent with dimerization of the motif being necessary for signaling.

In B lymphocytes, MPYS can be tyrosine phosphorylated. The phosphorylated MPYS recruits SH2 domain-containing proteins. Human and mouse MPYS contain five conserved tyrosine residues in their C-terminal cytoplasmic tails. Three of these tyrosines, Y244, Y260, Y313, are predicted to bind SH2 domains when phosphorylated. The function of a tailless mutant (truncated at amino acid 182) was tested. This mutant was unable to mediate activation of ISRE expression and behaved as a dominant negative. Thus it too appears to require dimerization for function. To more specifically assess the function of tail tyrosines 244, 260 and 313, the effect of point mutation of each to phenylalanine was examined. Mutation of any one of the three tyrosines to phenylalanine led to decreased ISRE activation by MPYS. Immunoblotting analysis indicated that the level of expression of these mutants was equivalent to that of wt MPYS. It appears these tyrosines may play partially redundant signaling roles.

Whether MPYS binds to JAK-STAT pathway intermediaries was examined. It was found that MPYS is tyrosine phosphorylated when over-expressed in 293GT cells. The phosphorylated MPYS binds STAT1. MPYS also binds Tyk2, but not to JAK1 or JAK2. As a control, HA-Igβ, a signal transducer expressed in B cells, was expressed ectopically and found not to associate with Tyk2. The Box 1 mutant failed to associate with Tyk 2. Moreover, knockdown Tyk2 expression in 293GT cells decreased MPYS induced ISRE activation. Knockdown Tyk2 did not affect MPYS induced NFκB or IFNβ activation. Accordingly, it appears that like the Type I IFN receptor, dimeric MPYS activates the ISRE promoter via Box 1-mediated association with and activation of Tyk2 and C-terminal tail mediated association with and activation of STAT1. It appears that MPYS activates ISRE expression by a pathway that involves Tyk2 and STAT1, and activates INF expression by a distinct pathway likely involving TBK1-IRF3.

Study of a 293GT variant line, i.e., 293GT*, which has a stronger IFNβ-responses to MPYS, indicated that Box 1 is also required for MPYS induced IFNβ response in the 293GT* cells. However, among the C-terminal tail tyrosine mutants, only the Y244F mutation affected the MPYS-induced IFNβ response.

Unlike the Y260 and Y313 tyrosines, Y244 is very close to two conserved serine residues, S240 and S242. It was found that S242 is particularly important for the MPYS-induced IFNβ response. Thus, the Y244 mutation may affect some important function of S242 in IFNβ activation. These data show that distinct signaling pathways emanate from MPYS that utilize different elements in MPYS.

It is believed that the dimeric MPYS behaves like the Type I IFN αβ receptor to activate ISRE. The 293GT cells do not have endogenous MPYS. Yet, VISA induced strong IFNβ promoter activity in these cells. To further explore the functional relationship between VISA and MPYS, the dominant negative MPYS tailless mutant (MPYS-TL) was used in the IFNβ responsive 293GT* cells. MPYS had a strong IFNβ response in the 293GT* cells while the tailless mutant did not. Furthermore, the tailless mutant functioned as a dominant negative mutant that appeared to completely inhibit the WT MPYS induced IFNβ response.

VISA transfection induced a large IFNβ response which is only partially inhibited by the dominant negative MPYS mutant (about 50%). Unlike the MPYS and TBK1, which had a synergistic effect on IFNβ response, VISA and MPYS co-expression did not result in a synergistic response. These data indicate that VISA induced IFNβ response is largely independent of MPYS.

### Discussion

Signals leading to ISRE and IFNβ promoter expression emanate from MPYS via distinct pathways. MPYS activation of the IFNβ promoter occurs via a pathway that is dependent on the N-terminal Box 1 motif, as well as the C-terminal tail S242 of MPYS. It is believed that one of these sites mediates interaction of MPYS with TBK1, since it has been shown that MPYS is phosphorylated by TBK1 and TBK1 is a downstream mediate MPYS signaling of INFβ production.

In contrast, the “direct” MPYS signaling of ISRE promoter activation that is independent of intervening IFNβ production occurs by a distinct mechanism that appears similar to that employed by the Type I IFN αβ receptor. MPYS is constitutively dimeric and inducibly tyrosine phosphorylated, and associates with Tyk2 and STAT1, which mediate ISRE activation. Tyk2 binding and ISRE signaling require the Box 1 motif. This response also requires tyrosines located in the MPYS C-terminal tail, which are likely sites of STAT1 binding. Thus, it is believed that the activation of MPYS lead to stimulation of an IFN receptor-like signaling pathway that stimulates ISRE expression. MPYS also associate with VISA, and this association does not appear to require the CARD domain of VISA and appears to occur through its transmembrane.

Although MPYS induced IFNβ production does not require VISA, it is believed that VISA induced IFNβ is mediated by MPYS. It was found that while VISA stimulate greater than 200 fold increases in IFNβ promoter activity in 293GT cells, MPYS produced no more than about 10 fold increase. Furthermore, while 293GT cells do not express detectable endogenous MPYS expression, VISA is able to induce greater than 200 fold increase IFNβ promoter activity in these cells. It was also found that the VISA induced IFNβ response is only partially inhibited by the dominant negative MPYS mutant.

The in vivo data also indicate that MPYS and VISA are activated by distinct mechanisms to induce IFNβ responses. VISA knockout mice mount normal responses to intracellular dsDNA, yet MPYS enhances intracellular dsDNA induction of IFNβ responses. While VISA mediates both RIG-I and MDA5 signaling, MPYS is appears to be involved only in RIG-I mediated signaling. MPYS is required for Listeria monocytogenes induced IFNβ response while VISA is not required for this response. These results indicate that MPYS is not a merely downstream adaptor protein for VISA but has a broader role, possibly serving as a PRR that recognizes pathogens or B-DNA through its LRR domain, and relays signals via Tyk2-STAT1-ISRE and TBK1-IRF3—IFNβ pathways, thus promoting innate defense.

Results also show that, while independent of VISA, MPYS functions at the crossroads of multiple innate signaling pathways. These newly discovered roles of MPYS by the present inventors in viral infection and mpys chromosomal location at 5q31 can be used a determinant in susceptibility to infection.

MPYS stimulates IFNβ expression in response to RNA, DNA viruses, and intracellular bacterial infection. It has been shown that majority of MPYS is found in mitochondria or endoplasmic reticulum, where it is believed to function as a downstream adaptor for VISA (also known as MAVS, IPS-1 and CardIf), activating production of IFNβ via interaction with TBK1/IRF3. It has been shown that MPYS can activate interferon-stimulating response elements (ISRE) independent of its stimulation of Type I IFN production, indicating that MPYS activates at least two distinct signaling pathways. The present inventors have discovered that MPYS signaling of ISRE expression occurs via interaction of its N-terminal juxtamembrane PxxPxP Box 1 motif with the tyrosine kinase Tyk2, and interaction of its C-terminal cytoplasmic domain with STAT1. Consistent with its occurrence as a homodimer, both Box 1 and C-terminal tail mutants display dominant negative activity for ISRE signaling. MPYS Box 1 motif is required for both MPYS-mediated IFNβ and ISRE activation. However, Tyk2 knockdown inhibits only the ISRE. C-terminal tail Y260 and Y313 are needed only for ISRE activation. It was also found that VISA can activate IFNβ in the absence of MPYS expression and function. Thus MPYS has a broader role in innate immunity and activates independent signaling pathways to stimulate ISRE and IFNβ expression.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. An isolated peptide having at least 80% homology with the sequence of SEQ ID NO:1.

2. The isolated peptide of claim 1, wherein said peptide has at least 90% homology with the sequence of SEQ ID NO:1.

3. The isolated peptide of claim 1, wherein said peptide has at least 95% homology with the sequence of SEQ ID NO:1.

4. The isolated peptide of claim 1, wherein said peptide has at least 99% homology with the sequence of SEQ ID NO:1.

5-9. (canceled)

10. A method for identifying a compound with a potential for treating a clinical condition in which a cell expressing MPYS is required for maintenance of the clinical condition comprising:
contacting a test compound with a protein or a cell comprising a protein having at least 80% homology with a sequence of a peptide of SEQ ID NO:1; and
identifying a compounds that modulates the protein activity,
wherein the protein activity modulation is indicative that the compound has a potential to treat the clinical condition in which a cell expressing MPYS is required for maintenance of the clinical condition.

11. The method of claim 10, wherein increase in the protein activity is indicative that the compound has a potential for treating the clinical condition.

12. The method of claim 11, wherein the clinical condition comprises tumor, inflammation, autoimmunity, transplantation or a combination thereof.

13. The method of claim 11, wherein tumor comprises leukemia, lymphoma or mastocytoma.

14. The method of claim 10, wherein decrease in the protein activity is indicative that the compound has a potential for treating the clinical condition.

15. The method of claim 14, wherein the clinical condition comprises immunodeficiency.

16-23. (canceled)

24. A method for reducing inflammation associated with a clinical condition in a subject, said method comprising administering to the subject in need of such a treatment a compound that inhibits MPYS.

25. The method of claim 24, wherein the clinical condition comprises a bacterial infection, a viral infection, or a combination thereof.


SEQUENCE LISTING (6 sequences)

SEQ ID NO: 1 - DNA, 1140 bp, Homo sapiens
atgccccactccagcctgcatccatccatcccgtgtcccaggggtcacggggcccagaaggcagccttggttctgctgagtgcctgcctggtgaccctttgggggctaggagagccaccagagcacactctccggtacctggtcctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctcttga

SEQ ID NO: 2 - protein, 379 aa, Homo sapiens [sequence omitted]

SEQ ID NO: 3 - DNA, 23 bp, Artificial Sequence
  Primer Sequence
cagcaattttcagtgtcagaagc

SEQ ID NO: 4 - DNA, 21 bp, Artificial
  Reverse Primer Sequence
tcatcctgtccttgaggcagt

SEQ ID NO: 5 - DNA, 24 bp, Artificial Sequence
  HPRT1 Forward Primer Sequence
ggacaggactgaaagacttgctcg

SEQ ID NO: 6 - DNA, 24 bp, Artificial
  HPRT1 Reverse Primer Sequence
tccaacaaagtctggcctgtatcc